(19) United States
(12) Patent Application Publication
(10) Pub. No.: US 2026/0224877 A1
(43) Pub. Date: Aug. 6, 2026

(54) GUIDE ELEMENT SYSTEM FOR INTRAVASCULAR ACCESS

(71) Applicant: VenoCare, Inc., Doral, FL (US)

(72) Inventors: Raul LEYTE-VIDAL, Doral, FL (US); Amir BELSON, Savyon (IL); Paul FENTON, Doral, FL (US); Daniel PICO, Doral, FL (US)

(21) Appl. No.: 19/152,437

(22) PCT Filed: Feb. 2, 2024
(86) PCT No.: PCT/US2024/014341

§ 371 Date: Jul. 30, 2025

Related U.S. Application Data
(60) Provisional application No. 63/482,901, filed on Feb. 2, 2023.

(51) Int. Cl. A61M 39/02; A61B 17/34; A61M 25/00; A61M 25/01; A61M 25/06

(52) U.S. Cl. CPC A61M 39/0247; A61M 25/0052; A61M 25/0068; A61M 25/0097; A61M 25/0108; A61M 25/0606; A61B 17/3423; A61M 2039/0258; A61M 2039/0279; A61M 2039/0291

(57) ABSTRACT

A guide element for an intravascular access device that may include an intravascular access device having a needle inside of a catheter lumen and a working space between an exterior surface of the needle and an interior surface of the catheter, the working space configured to receive one or more tools therethrough, and a guide element having a distal end and a proximal end separated by one or more annular elements, the guide element configured to be distally advanced from the intravascular access device inside of a blood vessel, wherein the one or more annular elements is configured to control at least a distal end of the guide element.

### CLAIM OF PRIORITY

This patent application claims priority to U.S. Provisional Patent Application No. 63/482,901, filed Feb. 2, 2023, titled “GUIDE ELEMENT SYSTEM FOR INTRAVASCULAR ACCESS,” which is incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Intravenous access, such as venipuncture, is a necessary element for several medical procedures. Venipuncture refers generally to the process of obtaining intravenous access for any one of a variety of purposes, including intravenous infusion, therapy, blood sampling, and the like. In the hospital, for example, venipuncture is commonly used to place a small intravenous catheter for delivering intravenous fluids, drug delivery, blood sampling and the like.

While venipuncture and other forms of vascular access in relatively healthy patients can be a simple matter, such access is often needed in patients who are not healthy and may have small, tortuous, collapsed, fragile, and/or difficult to locate arteries and/or veins. In such patients, venipuncture and other forms of vascular access can be very challenging, particularly to less experienced phlebotomists, paramedics, nurses, and other health care practitioners.

In addition to difficult access, many vascular catheter placement systems can result in accidental punctures and/or accidental needle contamination during or after placement of the intravascular catheter. Still further, some conventional catheter placement devices employ relatively complex deployment handle movements that lead to increases both cost and complexity. Additionally, conventional handle placement and movements can obscure the presence and status of the needle and guide structure or guide element components of the tool, thus making use of the insertion tool less intuitive.

For these reasons, it would be desirable to provide improved methods, systems, and tools for deploying intravascular catheters using needles and guide structures. It would be particularly desirable to provide simplified deployment systems and assemblies having fewer components and, even more desirably, to provide components which are clearly visible to the user and configured to be utilized and manipulated in a straightforward, intuitive manner. At least some of these objectives will be met by the various embodiments that follow.

### SUMMARY OF THE DISCLOSURE

In general, an intravascular access guide element for use in an intravascular access device may comprise an intravascular access device having a needle inside of a catheter lumen and a working space between an exterior surface of the needle and an interior surface of the catheter; a guide element having a shaft on a proximal end and a tip at a distal end, a portion of the distal end having an exterior shape adapted and configured to conform at least partially to the interior surface of the catheter and the exterior surface of the needle, the guide element configured to move distally and proximally along the working space and to be further distally advanced from the intravascular access device inside of and along a blood vessel; and wherein the tip on the distal most end of the guide element having a rounded distal end and a pre-set curvature where when the guide element is advanced so that the tip is outside of the working space the tip will begin to transition into the pre-set curvature; and further wherein the shaft is one of a wire, a braid or a combination of a wire and a braid.

This and any other examples described herein may further comprise one or more of the following. The tip may further comprise an open interior portion on the proximal end wherein the size and shape of the proximal end portion can be adapted and configured to receive the distal most end of the shaft. The shaft may be a wire having one or more deformations to increase the surface area of the wire within the tip proximal end portion. The shaft may be a braid. The shaft may be a wire and braid combination and the wire and braid a joined by one or more crimp zones. The tip on the distal most end of the guide element may be an atraumatic tip. A segment of the circumference of the needle may be removed or formed in a flat, convex, or concave shape to create a working space within the interior arc of the catheter interior surface and the needle surface and a portion of the distal end of the guide element can conform at least partially to the interior arc. A portion of the needle segment may be removed or formed forms to create a combined working and blood flashback space within the interior arc of the catheter interior surface and the needle surface.

In general an intravascular access device may comprise a handle having a proximal end and a distal end, a slot extending from the proximal end to the distal end; a catheter having a proximal catheter hub and a distal catheter lumen, the proximal catheter hub releasably engaged to the distal end of the handle; an access needle extending proximally from within a needle carrier through the catheter lumen, the access needle having at least one surface extending longitudinally along a perimeter portion of the access needle adapted to engage with a portion of a guide element, and a tissue-penetrating tip extending distally beyond the catheter lumen; a working space within the catheter along a length of the access needle at least one surface, wherein the working space is configured to facilitate passage of the guide element therethrough from a proximal end of the intravascular access device distally into a vessel; and a slide extending through the slot, the slide coupled to a proximal portion of the guide element of a first structure so that distal advancement of the slide advances a distal tip portion of the guide element of a second different structure from a position contacting a guide element cutout distally along the at least one surface of the access needle.

This and any other example described herein may further comprise one or more of the following. The at least one surface of the access needle may extend longitudinally down an exterior surface of the access needle. The working space may be defined by the needle surface and an interior surface of the catheter, wherein the working space may be selectively accessible from a proximal end of the intravascular access device. The working space may be configured to selectively open at the catheter distal end and wherein the working space may be configured to guide a tool or tool segment sliding therethrough. The access needle may comprise a plurality of flat surfaces, wherein each of the plurality of flat surfaces are associated with a separate working space within the catheter lumen. The intravascular access device may further comprise an actuation button coupled to the needle carrier, and an actuation element exerting a force on the needle carrier towards the proximal end of the handle, wherein when the actuation button is depressed the actuation element displaces the needle carrier and access needle toward the proximal end of the handle. The access needle can be retracted proximally towards the handle, and wherein the guide element is configured to remain in a distally advanced position.

The guide element first structure or second structure can be made entirely or partially of a metallic material, polymeric material, or a combination thereof. The guide element may comprise a plurality of segments, wherein one or more of the plurality of segments comprise a different material. The guide element first structure or second structure is a wire, a braid, or a combination that is formed entirely or partially of a metallic material, polymeric material, or a combination thereof. The access needle having at least one surface extending longitudinally along a perimeter of the access needle wherein the at least one surface is a segment of the circumference of the needle that is removed or formed in a flat, convex, or concave shape to create a working space within the interior arc of the catheter interior surface further wherein at least a portion of the surface is adapted and configured to conform to an outer surface of the guide element distal end or guide element tip.

The intravascular access guide element may further comprise one or more a radio opaque marker bands extending along the longitudinal axis of the catheter and within the sidewall of the catheter, each of the one or more radio opaque markers having a rectangular body and a beveled tip, wherein the beveled tip is directed towards the distal most end of the catheter. Each one of the one or more radio opaque markers is evenly spaced from each of the other one or more radio opaque markers about the circumference of the catheter. The intravenous access device may further comprise one or more a radio opaque marker bands extending along the longitudinal axis of the catheter and within the sidewall of the catheter, each of the one or more radio opaque markers having a rectangular body and a beveled tip, wherein the beveled tip is directed towards the distal most end of the catheter. Each one of the one or more radio opaque markers may be evenly spaced from each of the other one or more radio opaque markers about the circumference of the catheter.

The exterior surface of the needle may be flat, and a portion of the guide element tip distal end conforms to the flat portion of the needle, or the exterior surface of the needle may have a channel and a portion of the guide element tip distal end conforms to the shape, curvature or orientation of the channel. The access needle having at least one surface extending longitudinally along a perimeter portion of the access needle adapted to engage with a portion of a guide element includes a flat portion and a portion of the guide element distal tip conforms to the flat portion of the needle or the at least one surface of the needle has a channel, and a portion of the guide element tip distal end conforms to the shape, curvature or orientation of the channel. The guide element may be molded onto a distal segment of the shaft. A distal segment of the shaft may be affixed within the guide element.

In general, a guide element for use with an intravascular access device may comprise an intravascular access device having a needle inside of a catheter lumen and a working space between an exterior surface of the needle and an interior surface of the catheter, the working space can be configured to receive one or more tools therethrough, a guide element may have a distal end and a proximal end separated by one or more annular elements, the guide element can be configured to be distally advanced from the intravascular access device inside of a blood vessel, wherein the one or more annular elements may be configured to control at least a distal end of the guide element.

In some examples, the intravascular guide element may also have at least two annular elements, wherein a first annular element can be configured to control a direction of travel of the guide element distal end. The distal end of the guide element can be an atraumatic tip. The distal end of the guide element can be a formed distal tip. The guide elements can be comprised of a plurality of segments. The intravascular guide element may further comprise at least four annular elements. The needle may have a modified flat, convex, or concave surface wherein the working space is defined by an interior arc of the catheter interior surface and the modified surface of the needle. The distal tip may further comprise one or more deployment slits aligned with the working space. The catheter distal tip may further comprise one or more conforming segments, wherein a distal tip of the guide element can be configured to engage the one or more confirming segments.

In general, an intravascular access device may include a handle having a proximal end and a distal end, a slot extending from the proximal end to the distal end. A catheter may have a proximal catheter hub and a distal catheter lumen, the proximal catheter hub can be releasably engaged to the distal end of the handle. An access needle extending proximally from within a needle carrier through the catheter lumen, the access needle having at least one modified surface extending longitudinally along a perimeter of the access needle, and a tissue-penetrating tip may extend distally beyond the catheter lumen. Also included may be a working space within the catheter along a length of the access needle at least one modified surface with flat, convex, or concave type surfaces or, wherein the working space is configured to facilitate passage of one or more tool therethrough from a proximal end of the intravascular access device distally into a vessel. A slide can extend through the slot, the slide may be in communication with a proximal end of the guide element so that distal advancement of the slide advances a distal tip portion of the guide element from a position within a guide element cutout distally along the modified surface of the access needle.

In some examples, the modified surface of the access needle extends longitudinally down an exterior surface of the access needle. The at least one tool comprises a guide element. The working space may be defined by the needle flat surface and an interior surface of the catheter, wherein the working space is selectively accessible from a proximal end of the intravascular access device. The working space can be configured to selectively open at the catheter distal end and wherein the working space is configured to guide a tool or tool segment sliding therethrough. The intravascular access device may further include an access needle lumen, wherein the guide element may comprise a plurality of annular elements between a distal end and proximal end, wherein one or more of the annular elements may extend through the access needle lumen. The intravascular access device may further include a hemostasis valve having longitudinal channels around a perimeter of the homeostasis valve, wherein the hemostasis valve can be disposed within the proximal catheter hub. The access needle comprises a plurality of flat surfaces, wherein each of the plurality of flat surfaces can be associated with a separate working space within the catheter lumen.

In some examples, the intravascular access device may further include an actuation button coupled to the needle carrier, and an actuation element exerting a force on the needle carrier towards the proximal end of the handle, wherein when the actuation button is depressed the actuation element displaces the needle carrier and access needle toward the proximal end of the handle. The access needle is retracted proximally towards the handle, and wherein the guide element may be configured to remain in a distally advanced position. The intravenous access device may further include a spool of guide element in communication with the proximal end of the intravascular device, wherein a length of the guide element can be contained within the spool. The guide element may be made entirely or partially of a metallic material, polymeric material, or a combination thereof The guide element may comprise a plurality of segments and or layers, wherein one or more of the plurality of segments and/or layers may comprise a different material.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a perspective view from a distal end of an intravascular access device with an example of a guide wire extended through a catheter along an exterior surface of a needle with a guide element advanced beyond the distal tip of the needle, as described herein.

FIG. 2 shows a detailed perspective of a distal portion of the intravascular access in FIG. 1 including an example the atraumatic tip formed by the guide element beyond the distal end of the needle.

FIG. 3 is another perspective view of the distal end of an intravascular access device in the stowed or retracted configuration with the guide element seated in the notched tip of the intravascular catheter and the tissue penetrating tip exposed for use.

FIG. 4 shows another example of a distal segment of an intravascular access device with an example of a polymeric guide element distal tip illustrated in a transparent manner such that a metallic nitinol wire segment of the guide wire is arranged therein.

FIG. 5A and FIG. 5B show examples of access needles in a perspective view including detailed views and cross sectional features as described herein.

FIG. 6A to FIG. 6C are detailed top plan views of a section of the needle and guide element illustrating different needle-guide element configurations, as described herein.

FIG. 7 is a cross section of the distal portion of a needle and guide element in contact with one another inside of the catheter.

FIG. 8A and FIG. 8B are perspective views of exemplary guide elements from a proximal end including an atraumatic distal tip and guide element features adapted for engagement with a guide wire.

FIG. 9A to 9C show side elevation views of a guide wire and guide element distal segment including both an atraumatic configuration and a linear or stowed configuration with additional detailed view of the guide element in a cross section to expose a braided guide element interior.

FIG. 10 is a perspective view of a guide element with a section removed from view to expose another example of a braided interior comprising annular elements with various material properties, as described herein.

### DETAILED DESCRIPTION

An intravascular access device may include a needle at least partially disposed within a catheter. The needle can be configured to puncture and traverse one or more layers of tissue until access to a blood vessel is achieved. The catheter can advance through one or more layers of tissue as the needle is advanced accordingly. Once inside of a vessel, it may be desirable for the catheter to advance beyond the needle into the vessel. A guide element generally positioned between an exterior surface of the needle and an interior surface of the catheter may be selectively manipulated (e.g., advanced) ahead of the catheter distal tip to aid in guiding a placement of the catheter in the vessel distal to the point of insertion. The guide element may have one or more features providing increased function, control, and safety.

An intravascular access device, as described herein, may comprise a working space or working channel created by a needle having a semi-circular cross-sectional geometry. Current intravascular access devices provide for concentric circular cross-sectional geometry between the needle and catheter (e.g., a tubular needle within a tubular catheter). The intravascular access devices described herein provide a needle with a semi-circular cross-sectional geometry. For example, a needle having a circumference comprising an arc and a generally flat, concave or convex segment. In some examples, the arc portion of the needle circumference can be less than 360 degrees, less than 350 degrees, less than 340 degrees, less than 330 degrees, less than 320 degrees, less than 310 degrees, less than 300 degrees, less than 270 degrees, less than 180 degrees, or more. In some examples, the flat, concave, convex surface of the needle defines a discontinuous circular circumference (e.g., a circle with a segment removed). In some examples, the discontinuous segment on the perimeter of the needle may relate to a removed or formed minor segment from an otherwise complete circumference.

Dimensions of the working space may be an area of the removed or formed minor segment of the needle. For examples, considering a cross-section of the needle and catheter at any point along their length, an area of the working space may be calculated as the difference between the area of the needle and the area of the circular cross-section of the catheter. For example, at any cross section of the catheter an area may be calculated as itr2, r being a radius of the catheter circular cross-section; and an area of the needle may be calculated as itr2−the area of the removed or formed minor segment. Accordingly, the working space may be the difference between the area of the interior of the catheter and the area of the needle.

The working space may be configured to accommodate one or more tools (e.g., guide elements). In some examples, the working space between the exterior of the needle and the interior surface of the catheter may be configured to accommodate a guide element, as described herein. In some examples, the working space may be configured to accommodate procedurally related tools. For example, a tool may be selectively passed into and/or through the working space from a proximal end of the intravascular access device to a distal end or distal tip of the intravascular access device (e.g., a distal tip of the catheter).

The working space may be configured to accommodate the administration of one or more therapeutics during an intravascular access procedure. For example, the distal tip of the needle may first penetrate and traverse biological tissue until entering a vessel. The catheter of the intravascular access device may traverse the biological tissue with the needle until the distal tip of the catheter is within the vessel. The operator may then introduce a tool from a proximal end of the intravascular access device accessible outside of the patient's body and advance the tool through the intravascular access device such that the tool passes or slides within the working space between the needle exterior surface and the interior surface of the catheter until it is functionally deployed within the vessel as allowed by the positioning of the catheter distal tip. The operator may engage a proximal end of the tool and/or a proximal end of the intravascular access device to control the tool within the blood vessel. After a procedure is complete, the operator may retract the tool through the working space allowing the intravascular access device to remain in position within the vessel.

According to any example described herein, it may be useful to guide an intravascular access device into and/or through a patient's vasculature. For example, the intravascular access device may first penetrate and traverse biological tissue until entering a vessel. The catheter of the intravascular access device may need to be advanced beyond the initial placement into the vessel facilitated by the needle distal tip. Accordingly, a guide element may be deployed in advance of the catheter deployment to aid in the routing and positioning of the catheter through distal areas of the vasculature. A guide element, as described herein may include a distal end and a proximal end separated by a length of guide element body configured to pass through an intravascular access device having a working space between the needle and an interior of the catheter.

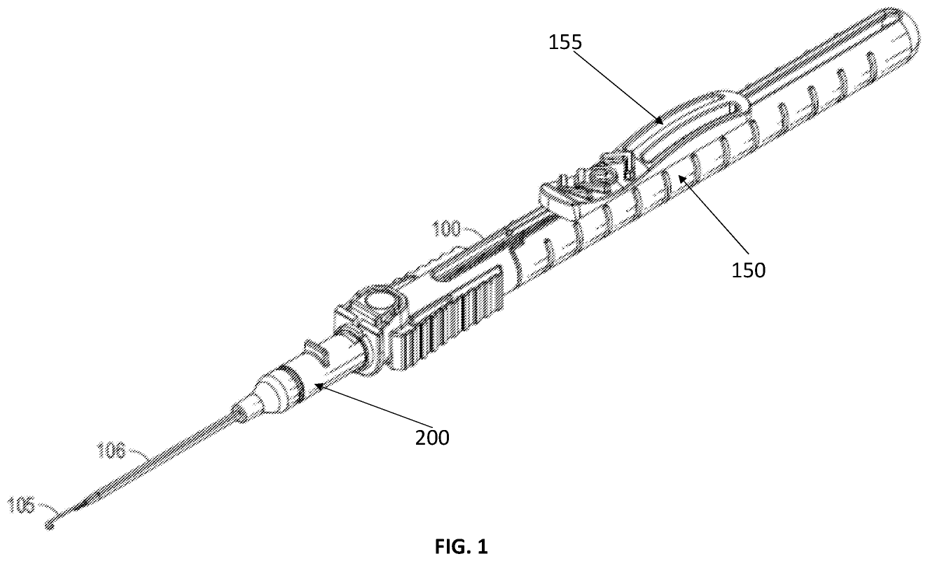

FIG. 1 illustrates an example of an intravascular access device 100 having a handle 150 and a slider 155 adapted to control the deployment of the guide element 105 through a lumen extending through the catheter 106 and along the needle exterior surface positioned within the catheter lumen. An example of a guide element feature is illustrated where the guide element 105 is generally curled and formed with an atraumatic distal end beyond the needle tip as it may be within a vessel of a patient when the guide element is deployed. In some examples, advantage of an atraumatic tip or distal end of the guide element, as described herein, is to prevent unintended perforation of the vessel by the guide element or other adverse events such as snagging of the guide element distal end by the patient's vascular anatomy.

Guide elements described herein may have a distal end 108 configured to engage an interior of a vessel. For example, the guide element distal end 108 may comprise the distal tip (e.g., 121, 122, etc.) and a segment of the guide element body proximal to the tip. Referring to FIG. 1, the guide element 105 may include the curled atraumatic distal end 108 configured to advance through a vessel and maintain the curled geometry as it is advanced through the vessel. In some examples, the distal end of the guide element may apply a force against an interior of the vessel to open or otherwise facilitate passage of the catheter through a collapsed or partially collapsed vessel.

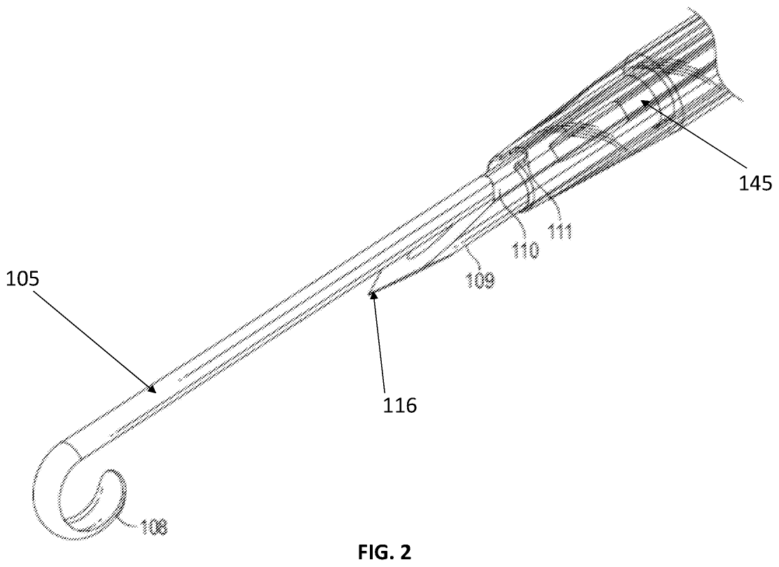

FIG. 2 illustrates additional details of the guide element 105, shown in FIG. 1 with the guide element distal end 108 curled forming an atraumatic tip. In some examples, the guide element can be configured to transition from a generally linear configuration when stowed or in a ready to use configuration then may automatically confirm to the atraumatic distal end 108 as the guide element 105 is advanced distally from the intravascular access device 100. In FIG. 2, the guide element is shown as advancing from the needle 109. The needle 109 may have an aperture 110 extending therethrough from the needle distal tip to a proximal end of the need (not shown). The guide element 105 may be configured to pass or be advanced through the needle aperture into the vasculature of the patient.

In some examples, the guide element 105 may initially be disposed in one or more retracted or ready configurations throughout the intravascular access device 100. When deployed, as shown in FIG. 2, the guide element distal end 108 curls beyond the needle tissue penetrating tip 116 as it is advanced from the catheter 106 and from the distal slit or notch 111 of the catheter distal end. The notch 111 at the catheter distal end can be configured to receive a portion of the guide element when the guide element 105 is in a stowed configuration to provide a seamless or smooth transition from the tissue penetrating tip to the catheter body.

FIG. 2 further illustrates the distal end of the intravascular access device 100 and the guide element a distal end 108 curling over the needle distal tip. The guide element is extending outward from the working space between the catheter 106 interior surface and the needle exterior surface. The needle flat surface, in this example, extends along the needle body to the needle distal tip and a catheter distal tip slit 111 is shown in an open position biased opened by the advanced guide element passing therethrough.

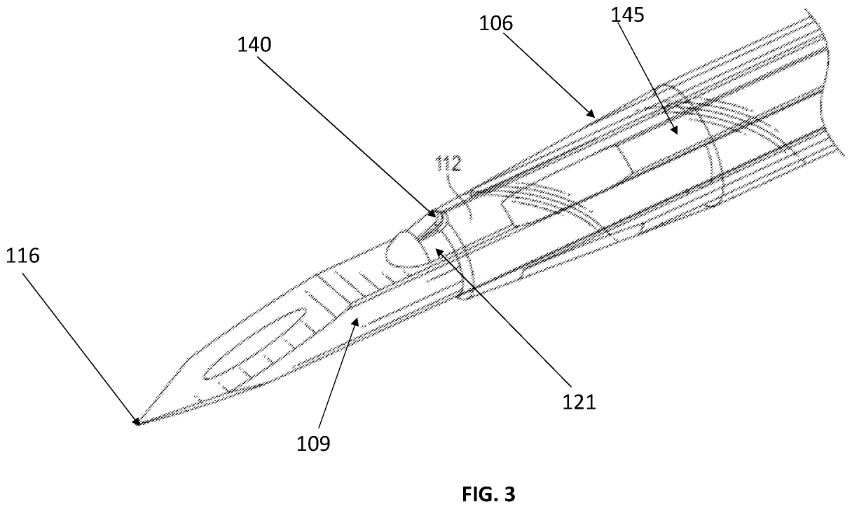

FIG. 3, the guide element 105 is fully retracted into the working space 112 between the catheter and the needle 109. In some examples, the guide element 105 may be generally linear from the distal end to a proximal end and may be configured to slide through the intravascular access device 100 through the working space 112 between the needle exterior surface and the catheter interior surface. The guide element distal tip 121 is seated in the catheter distal end with the guide element docking feature 140 is seated into the catheter notch. The view in FIG. 3 also includes some examples of positional markers or radiopaque elements 145 in the catheter 106 configured to indicate the position of the catheter within the patient.

In some examples, devices described herein can be configured to access a vessel with the needle 109 including the stowed guide element 105 in the catheter distal end notch 111. When the vessel has been accessed, the guide element 105 may be deployed to support advancement of the catheter and positioning of the catheter in the vessel before the guide element and needle are retracted and the catheter is left in place within the patient.

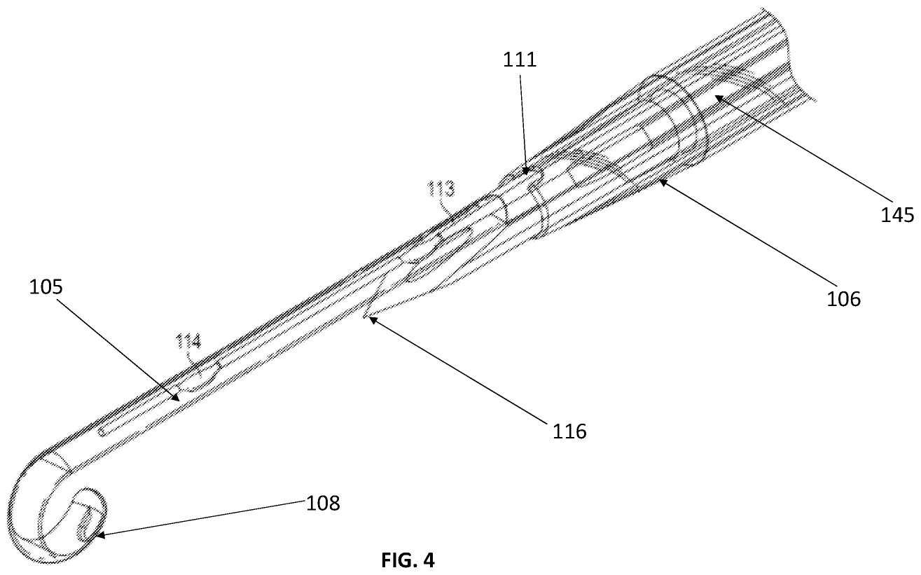

In FIG. 4, a guide element 105 may be attached to a polymeric monofilament or metallic wire 113 by over-molding or another method. In this example, a polymeric monofilament or metallic wire 113 is constructed or formed in the distal section 114 with features to facilitate a mechanical lock and permanent attachment of the guide element to the polymeric monofilament or metallic wire. The metallic wire (e.g., guide wide) may comprise one or more features such as 114 with an irregular geometry compared to the length of wire extending proximally therefrom. The engagement features 114 can be configured to support the connection between the guide wire 113 and the guide element 105 as it may be molded onto the distal end of the guide wire 113.

As shown in FIG. 4, the guide element 105 is shown in a substantially transparent manner to allow exposure of the engagement feature 114 positioned within the guide element 105 that is mold onto the distal end or portion of the wire 113. The engagement feature example 114 is a flat paddle-style feature that would retrain the connection between the guide element 105 and the wire 113 to prevent the guide element 105 from separating off the wire after it is molded thereon.

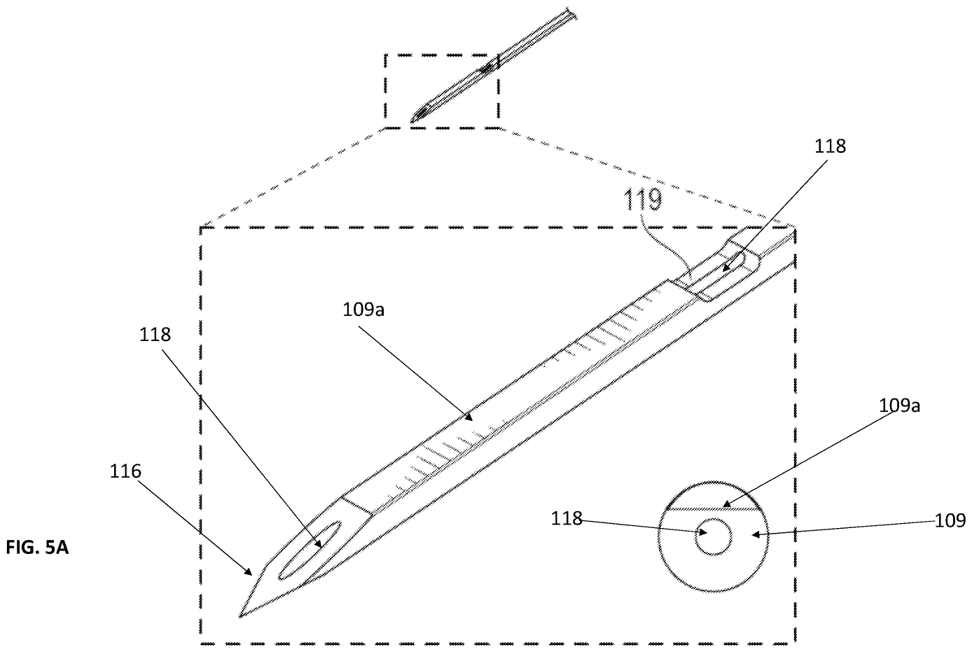

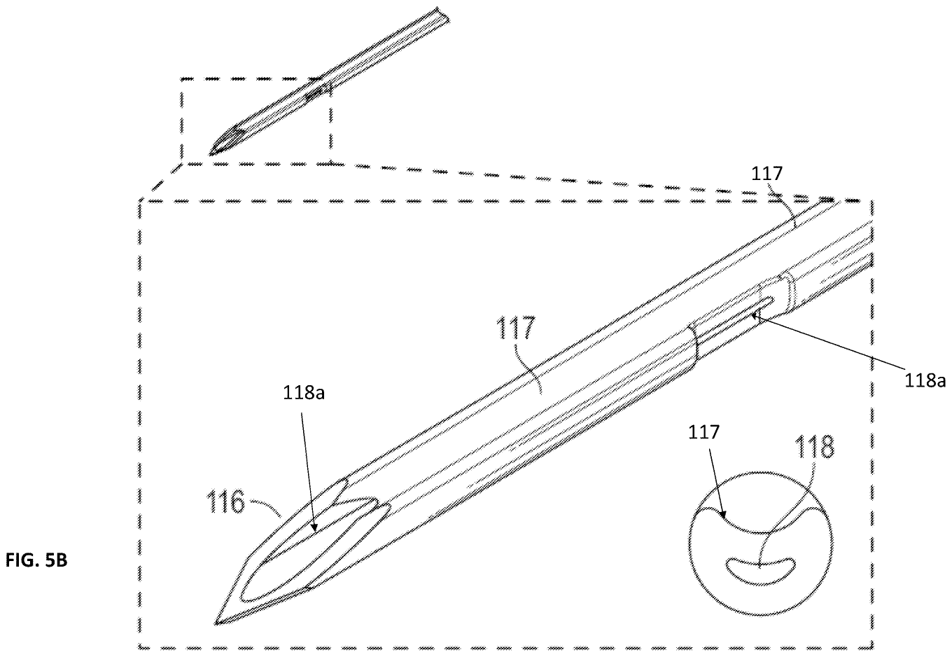

FIG. 5A and FIG. 5B are closeup views of the distal end of the intravascular access device needle illustrating examples of removed or formed needle segments along the circumference and the cross-sectional area of the needles as described herein. Referring to FIG. 5A, needle 109 is shown with the detailed expanded view highlighting a flat surface 109a along the needle exterior. The flat surface allows for an increased volume of space or working space within the catheter 106 when the needle is positioned therethrough. For example, the needle flat surface allows for a circular segment shaped working space within the catheter between the catheter interior surface and the needle exterior surface allowing the guide element to pass therethrough. The tissue penetrating tip 116 can be seen with the needle lumen 118 open through the bevel surface adjacent to the tissue penetrating tip 116. The proximal end of the needle is not shown.

Needle configurations described herein can be adapted to allow the guide element to be selectively advanced such that the operator may advance the guide element distally from the intravascular access device through the needle aperture, or through the working space, or some combination of both. In this configuration, the needle lumen 118 remains patent to allow flashback and visualization of blood during intravascular access through the side vents 119.

In some examples an alternative to the flat surface may be a formed needle segments with a channel 117 with a U-shape or C shape cross-section and thus merging the working space and needle lumen 118a into one longitudinal open cross-sectional area.

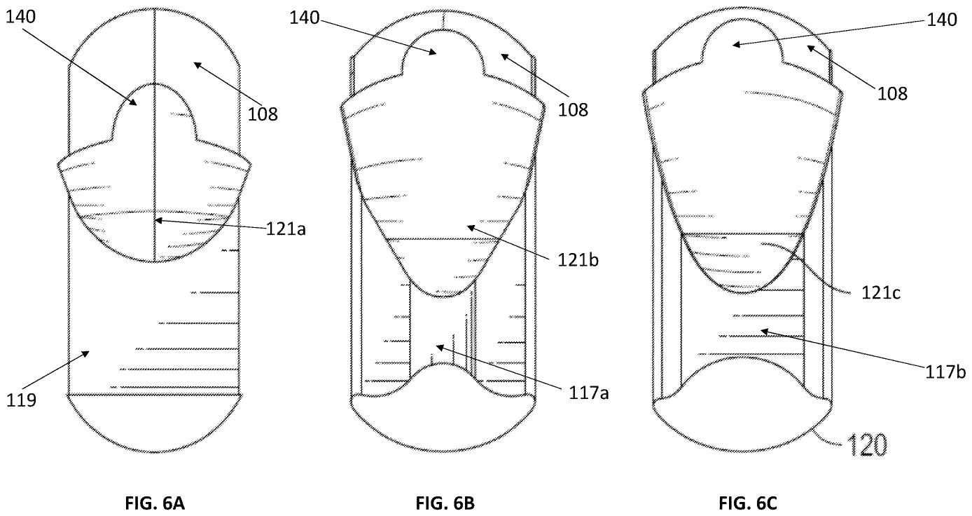

FIG. 6A to FIG. 6C further illustrate examples of the needle cross section geometry including perspective views from the top of the guide element distal end 108 including guide element distal tips 121a, 121b, and 121c adjacent to the needle exterior surface. Looking at the cross section of the distal position of the needle highlights examples of the needle configuration (e.g., adapted surface). Referring to FIG. 6A, the flat surface 119 is visible. Although the contacting surface of the guide element is not shown, FIG. 6A may also include a guide element with a conforming flat surface to contact and slide along the needle flat surface 119. Referring to FIG. 6B, the channel 117a is visible and may extend along the entire length of the needle or less than the entire length of the needle. The channel 117a may have a width and depth that the guide element may conform to or substantially conform to. For example, the guide element tip 121b may have a surface adapted to seat or conform with the channel 117a. Referring to FIG. 6C, the channel 117b formed into the needle body. Similar to FIG. 6B, the distal tip 121c illustrated in FIG. 6C may have a conforming surface adjacent to the channel 117c to promote the routing of the guide element and increase the amount of working space within the catheter for the guide element to operate or be stowed within. Each of the examples illustrated in FIGS. 6A, 6B, and 6C include the guide element docking feature 140 adapted to engage the catheter distal end (e.g., notch 111 not shown).

In some examples, the guide element surface contacting the needle exterior surface may include a formed distal tip 121. The guide element distal tip may be defined by a portion of the guide element (e.g., the distal end or distal end segment) or by a formed distal end. Formed distal ends of a guide element described herein may include distal ends that are attached to a distal tip of the guide element. These formed distal ends 121 may be molded with the guide element during initial manufacture or may be affixed to the guide element at some time after. In some examples, the formed distal end may be a modified distal tip such that the distal tip of the guide element is modified to a shape or configuration.

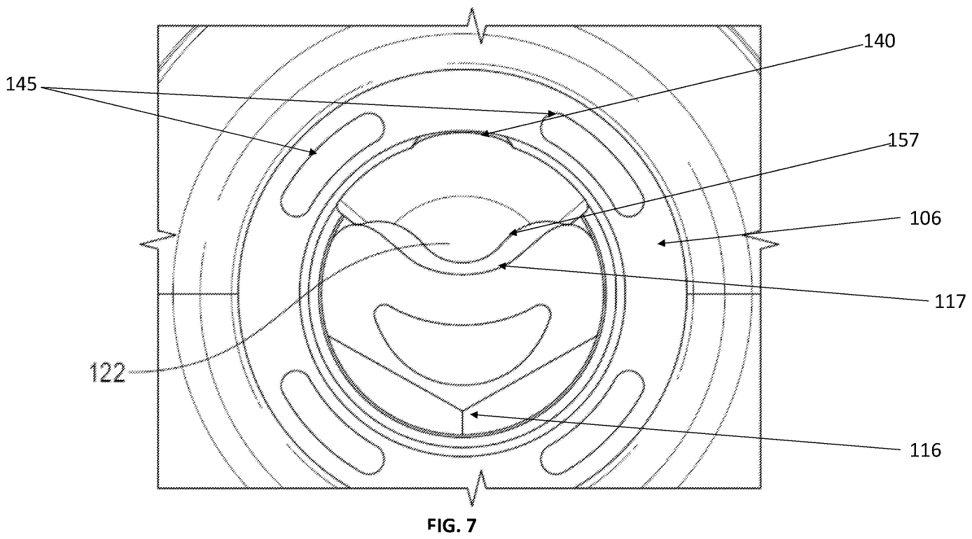

FIG. 7 provides a cross section of a profile view from the distal end of the needle 116, guide element formed distal tip 122, radiopaque markers 145 within the catheter 106. In this example, the guide element contacting surface 157 is shown formed and substantially conforming to the channel 117. For example, the guide element surface 157 essentially completing the needle distal end geometry to a cylinder or tubular shape by seating in the channel 117. Additionally, the guide element distal tip 122 is shown tapered from near the distal end of the needle (e.g., near the needle tip) to the distal end of the catheter. In this configuration, there is a reduced impact of the distal end of the intravascular access device as it penetrates and traverses the biological tissue and vessels of a patient's anatomy, lowering the penetration force. In some examples, the distal segment of the guide element is molded, welded, shaped, or configured for a particular function.

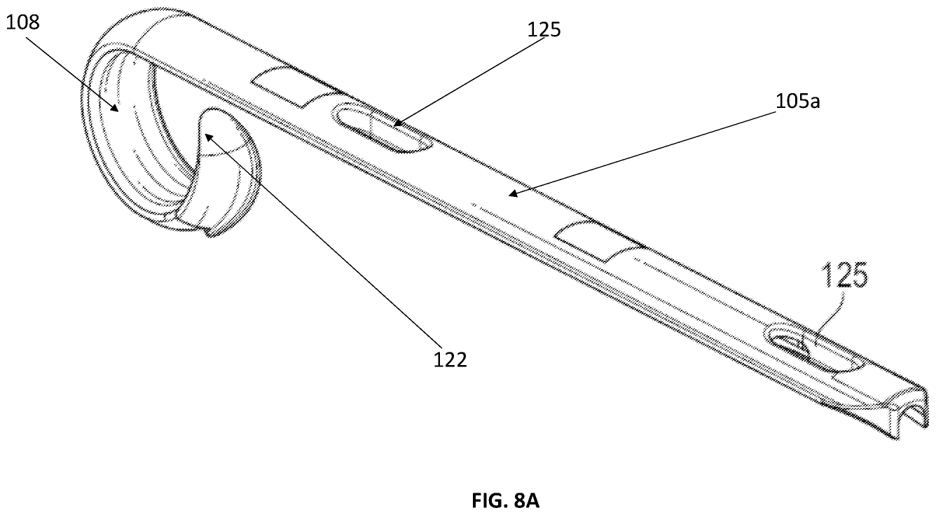

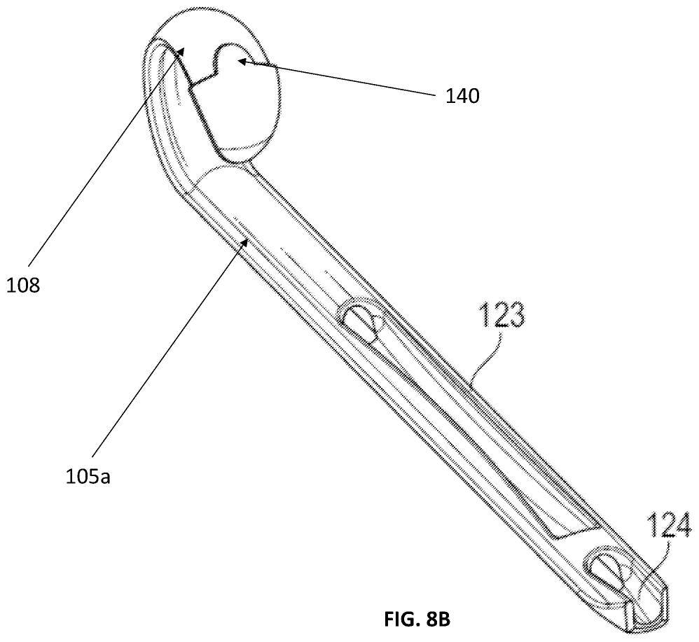

FIG. 8A and FIG. 8B, an illustration of a guide element alone without any other components of the intravascular access device. The molded guide element 105a may be manufactured first and separately then affixed to a polymeric monofilament or metallic wire. Referring to FIG. 8A, openings 125 may be configured to receive an adhesive or another coupling mechanism to affix the guide element 105a to the wire (not shown). For example, after the guide element 105a is formed, a wire may be introduced through the guide element 105a and then affixed using openings 125 and/or 123 in FIG. 8B. Each of these openings can be used to facilitate the attachment of the polymeric monofilament, metallic wire, or combination thereof, the parts remain interlocked after assembly via bonding, welding, or any other particular assembly process to the guide element 105a.

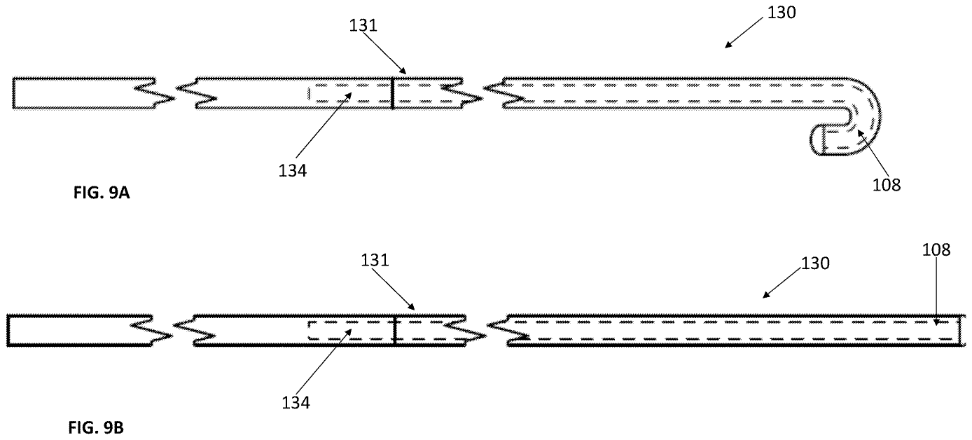

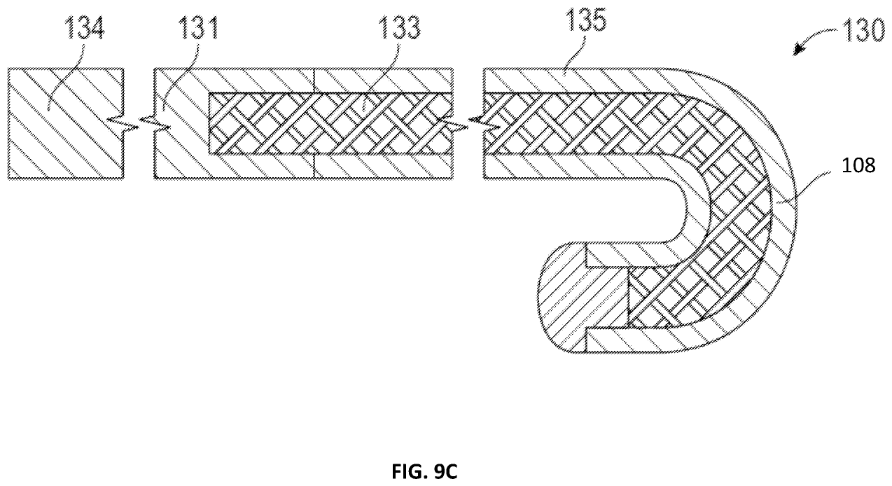

In some examples, the guide element may have a braided core or interior. FIGS. 9A, 9B, and 9C illustrates a guide element 130 for an intravascular access device whereby the guide element is a composition of annular elements with differing material properties.

Referring to FIGS. 9A and 9B, a guide element 130 is shown in a curled configuration (e.g., deployed configuration) and stowed configuration in FIG. 9B with the guide element substantially linear. The guide element proximal end 131 can be relatively stiff to provide pushability. In FIG. 9C, the distal end of the guide element 132 is soft and flexible to provide an atraumatic insertion into the vasculature, a braided inner core 133 to providing good strength in tensile crimped within the crimp zone 134 to the proximal end 131. The braid inner core is positioned within an outer sheath 135, providing a smooth surface during insertion into the vasculature. In this example, the looped or curled distal end of the element can be formed using preset methods, molding, dip coating, welding, or any other particular assembly process.

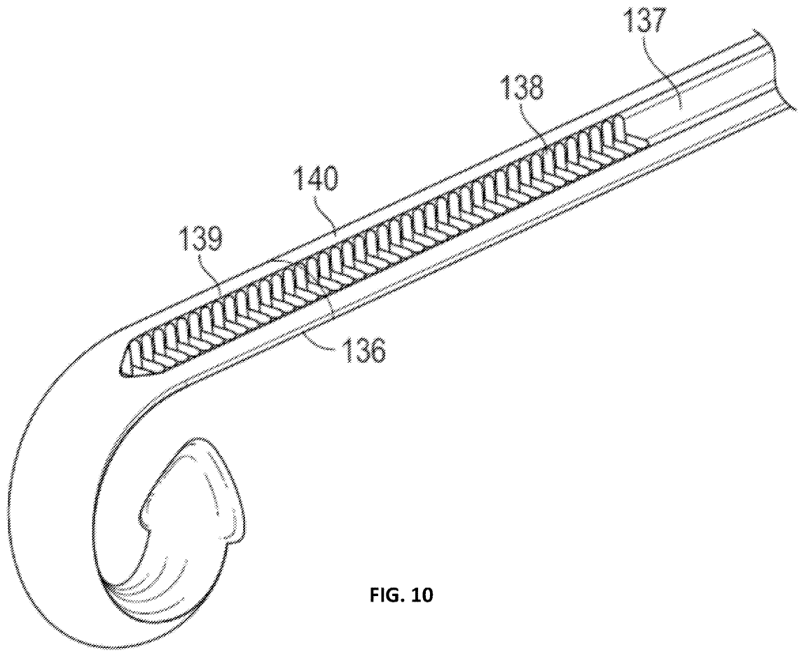

FIG. 10 illustrates an example of a guide element for an intravascular access device in an extended or advance configuration whereby the guide element 136 is a composition of annular elements with differing material properties. In this example, a polymeric monofilament or metallic wire 137 over-braided using a polymeric or metallic monofilament in a single or multi layered pattern 138. The braided polymeric monofilament or metallic wire 137 is over-molded 139 to form guide element 105. Whereas the braided segment of the guide element provides good strength in tensile for the guide element and relative stiffness to provide pushability. The braided polymeric monofilament or metallic wire 137 is positioned within an outer sheath 140 providing a smooth surface during insertion into the vasculature.

The guide element structures may be configured or have a geometry to compliment the working space, the needle formed or flat surface, the interior surface of the catheter, the needle aperture, the needle distal end, the needle distal tip, procedural parameters, the guide element function, guide element steerability, etc. For example, the guide element may be geometrically configured to conform to the needle exterior and the needle exterior may be curved such that the guide element cross-sectional geometry is concave to compliment the curved exterior of the needle. In some examples, the geometry of each of the annular elements may complement each other such that the annular elements maximize the volume occupied by the guide element in the intravascular access device. In some examples, the geometry of the annular elements may complement one another such that they promote optimized steerability or function of the guide element. In some examples a guide element may have two or more annular elements that can be generally cylindrical (e.g., circular cross-sections). In some examples, the dimensions of the guide element and/or each annular element may consider the function or deployment route through the intravascular access device. For example, an annular element may have a diameter between 0.005″ and 0.050″. In some examples, each annular element may have a width between 0.005″ and 0.050″. In some examples, the width of an annular element may be relative to the width of one or more other annular elements of the same guide element. In some examples, each annular element may have a different width. In some examples, more than one annular element may have the same width.

In some examples, the retracted guide element forms an atraumatic surface with the distal end of the intravascular catheter thus reducing the impact of the distal end of the intravascular access device as it penetrates and traverses the biological tissue and vessels of a patient's anatomy, lowering the penetration force. For example and as illustrated in FIG. 7, a formed distal tip 122 essentially completing the needle distal end geometry to a cylinder or tubular shape that is tapered from near the distal end of the needle (e.g., near the needle tip) to the distal end of the catheter.

Some examples of materials comprising the guide element, or a guide element segment may include nitinol, PEEK, or another material having sufficient rigidity and stiffness to support advancement through vasculature. Additionally, the material may have memory wherein a shape may be imparted into the material through molding or other forming means such that the shape is restricted in a retracted state and the shape is re-established after the shaped portion is advanced out of the IV device/system.

In some examples, the guide element and/or one or more of the guide elements components may comprise one or more materials. The materials can be of alloy or polymeric nature. In some examples, the material composition of the guide element may be different at different areas of segments along the length or at various segments of the guide element. For example, the distal end may comprise one or more materials configured to adapt, adjust, or otherwise change a shape or orientation of the distal segment. For example, the distal tip may be a first material, a segment adjacent to the distal tip may be a second material, and a segment adjacent thereto may be a third material. The second material may have properties that are different than the first and third materials causing the second material segment to change, confirm, or otherwise react to different environmental factors causing a predetermined or desirable change in the configurations and orientation of the guide element distal end. In some examples, a segment may contract at a faster rate in the presence of lower temperatures compared to other segments resulting in predetermined curves or bends at said segment. In some examples, a segment may contract at a faster rate in the presence of higher tension caused by the braiding compared to other segments resulting in predetermined curves or bends at said segment. In some examples, the guide element may have more than one annular segment and each annular segment may comprise a different materials and/or be comprised of materials having different attributes such as stiffness, malleability, hardness, conductivity, etc. For example, one annular element may comprise a material of sufficient hardness to be advanced through an occlusion without causing a bend or kink in the guide element as it is advanced therethrough, while a second annular element may have less stiffness that may bend if the guide element were advanced only using the softer annular element. In such a configuration, the soft sided annular element may act as a test element when advancing the guide element through vasculature and allow for testing of the amount of force necessary to advance the guide element through an occlusion.

There may be one or more variations, alternatives, constructions, compositions, and/or components described herein that can be used to modify an element, component, device, system, process, etc. of a guide element, intravascular access device, needle and/or an associated structure or process. Accordingly, any variation, description, example, element, component, process, method, method step, etc. described herein can be used as a modification, variation, and/or alternative to any element, device, system, composition, example, component, process, method, method step, etc. described in PCT application number PCT/US23/84427 filed on Dec. 15, 2023, entitled “HYPODERMIC NEEDLES AND METHODS OF MANUFACTURE”; and/or PCT application number PCT/US23/86107 filed on Dec. 27, 2023, entitled “INTRAVASCULAR ACCESS DEVICE” the entireties of which are incorporated herein.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical” , “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of or alternatively ”consisting essentially of the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An intravascular access guide element for use in an intravascular access device, comprising:
a. An intravascular access device having a needle inside of a catheter lumen and a working space between an exterior surface of the needle and an interior surface of the catheter;
b. A guide element having a shaft on a proximal end and a tip at a distal end, a portion of the distal end having an exterior shape adapted and configured to conform at least partially to the interior surface of the catheter and the exterior surface of the needle, the guide element configured to move distally and proximally along the working space and to be further distally advanced from the intravascular access device inside of and along a blood vessel; and
c. wherein the tip on the distal most end of the guide element having a rounded distal end and a pre-set curvature where when the guide element is advanced so that the tip is outside of the working space the tip will begin to transition into the pre-set curvature; and further wherein the shaft is one of a wire, a braid or a combination of a wire and a braid.

2. The intravascular access guide element of claim 1, the tip further comprising an open interior portion on the proximal end wherein the size and shape of the proximal end portion is adapted and configured to receive the distal most end of the shaft.

3. The intravascular access guide element of claim 2, wherein the shaft is a wire having one or more deformations to increase the surface area of the wire within the tip proximal end portion.

4. The intravascular access guide element of claim 2, wherein the shaft is a braid.

5. The intravascular access guide element of claim 2, wherein the shaft is a wire and braid combination and the wire and braid a joined by one or more crimp zones.

6. The intravascular access guide element of any of claims 1-5, wherein the tip on the distal most end of the guide element is an atraumatic tip.

7. The intravascular access guide element of any of claims 1-5, wherein a segment of the circumference of the needle is removed or formed in a flat, convex, or concave shape to create a working space within the interior arc of the catheter interior surface and the needle surface and a portion of the distal end of the guide element conforms at least partially to the interior arc.

8. The intravascular access guide element of any of claims 1-5, wherein a portion of the needle segment is removed or formed forms to create a combined working and blood flashback space within the interior arc of the catheter interior surface and the needle surface.

9. An intravascular access device, comprising:
a handle having a proximal end and a distal end, a slot extending from the proximal end to the distal end;
a catheter having a proximal catheter hub and a distal catheter lumen, the proximal catheter hub releasably engaged to the distal end of the handle;
an access needle extending proximally from within a needle carrier through the catheter lumen, the access needle having at least one surface extending longitudinally along a perimeter portion of the access needle adapted to engage with a portion of a guide element, and a tissue-penetrating tip extending distally beyond the catheter lumen;
a working space within the catheter along a length of the access needle at least one surface, wherein the working space is configured to facilitate passage of the guide element therethrough from a proximal end of the intravascular access device distally into a vessel; and
a slide extending through the slot, the slide coupled to a proximal portion of the guide element of a first structure so that distal advancement of the slide advances a distal tip portion of the guide element of a second different structure from a position contacting a guide element cutout distally along the at least one surface of the access needle.

10. The intravascular access device of claim 9, wherein the at least one surface of the access needle extends longitudinally down an exterior surface of the access needle.

11. The intravascular access device of claim 9 or claim 10, wherein the working space is defined by the needle surface and an interior surface of the catheter, wherein the working space is selectively accessible from a proximal end of the intravascular access device.

12. The intravascular access device of any claim 11, wherein the working space is configured to selectively open at the catheter distal end and wherein the working space is configured to guide a tool or tool segment sliding therethrough.

13. The intravascular access device of any of claims 9-12, wherein the access needle comprises a plurality of flat surfaces, wherein each of the plurality of flat surfaces are associated with a separate working space within the catheter lumen.

14. The intravascular access device of any of claims 9-13, further comprising an actuation button coupled to the needle carrier, and an actuation element exerting a force on the needle carrier towards the proximal end of the handle, wherein when the actuation button is depressed the actuation element displaces the needle carrier and access needle toward the proximal end of the handle.

15. The intravascular access device of claim 14, wherein the access needle is retracted proximally towards the handle, and wherein the guide element is configured to remain in a distally advanced position.

16. The intravenous access device of claim 9, wherein the guide element first structure or second structure is made entirely or partially of a metallic material, polymeric material, or a combination thereof.

17. The intravenous access device of any of claims 1-16, wherein the guide element comprises a plurality of segments, wherein one or more of the plurality of segments comprise a different material.

18. The intravenous access device of any of claims 1-16, wherein the guide element first structure or second structure is a wire, a braid, or a combination that is formed entirely or partially of a metallic material, polymeric material, or a combination thereof.

19. The intravenous access device of claim 9, the access needle having at least one surface extending longitudinally along a perimeter of the access needle wherein the at least one surface is a segment of the circumference of the needle that is removed or formed in a flat, convex, or concave shape to create a working space within the interior arc of the catheter interior surface further wherein at least a portion of the surface is adapted and configured to conform to an outer surface of the guide element distal end or guide element tip.

20. The intravascular access guide element of claim 1, further comprising one or more a radio opaque marker bands extending along the longitudinal axis of the catheter and within the sidewall of the catheter, each of the one or more radio opaque markers having a rectangular body and a beveled tip, wherein the beveled tip is directed towards the distal most end of the catheter.

21. The intravascular access guide element of claim 20, wherein each one of the one or more radio opaque markers is evenly spaced from each of the other one or more radio opaque markers about the circumference of the catheter.

22. The intravenous access device of claim 9, further comprising one or more a radio opaque marker bands extending along the longitudinal axis of the catheter and within the sidewall of the catheter, each of the one or more radio opaque markers having a rectangular body and a beveled tip, wherein the beveled tip is directed towards the distal most end of the catheter.

23. The intravenous access device of claim 22, wherein each one of the one or more radio opaque markers is evenly spaced from each of the other one or more radio opaque markers about the circumference of the catheter.

24. The intravascular access guide element of claim 1, wherein the exterior surface of the needle is flat, and a portion of the guide element tip distal end conforms to the flat portion of the needle, or the exterior surface of the needle has a channel, and a portion of the guide element tip distal end conforms to the shape, curvature or orientation of the channel.

25. The intravenous access device of claim 9, wherein the access needle having at least one surface extending longitudinally along a perimeter portion of the access needle adapted to engage with a portion of a guide element includes a flat portion and a portion of the guide element distal tip conforms to the flat portion of the needle or the at least one surface of the needle has a channel and a portion of the guide element tip distal end conforms to the shape, curvature or orientation of the channel.

26. The intravenous access device of any of claim 1, wherein the guide element is molded onto a distal segment of the shaft.

27. The intravenous access device of any of claims 1 to claim 8, wherein a distal segment of the shaft is affixed within the guide element.
